(19) United States
(12) Patent Application Publication
(10) Pub. No.: US 2026/0091270 A1
(43) Pub. Date: Apr. 2, 2026

(54) BOOSTING TIME-RELEVANT CONTENT IN A CONNECTED FITNESS PLATFORM

(71) Applicant: Peloton Interactive, Inc., New York, NY (US)

(72) Inventors: Shayak BANERJEE, Ardsley, NY (US); Vijay PAPPU, Jersey City, NY (US); Nilothpal TALUKDER, Poughkeepsie, NY (US); Shoya YOSHIDA, New York, NY (US); Arnab BHADURY, Vancouver, CA (US); Allison SCHLOSS, Brooklyn, NY (US); Jasmine PAULINO, Brooklyn, NY (US)

(21) Appl. No.: 19/111,994

(22) PCT Filed: Sep. 14, 2023
(86) PCT No.: PCT/US2023/074234

§ 371 Date: Mar. 14, 2025

Related U.S. Application Data
(60) Provisional application No. 63/406,400, filed on Sep. 14, 2022.

(51) Int. Cl. A63B 24/00; G06Q 10/04; G16H 20/30

(52) U.S. Cl. CPC A63B 24/0075; G06Q 10/04; G16H 20/30; A63B 2024/0081

(57) ABSTRACT

Systems and methods that enhance an exercise activity are described. In some embodiments, the systems and methods personalize certain time-based content for users/members to enhance their experience when exercising or navigating a connected fitness platform. For example, the systems and methods may utilize machine learning models to determine or predict a likelihood of engagement of timely content by users and ranks the content or provides recommendations to the users in response to the determined predictions.

### BACKGROUND

This application claims priority to U.S. Provisional Patent Application No. 63/406,400, filed on Sep. 14, 2022, entitled BOOSTING TIME-RELEVANT CONTENT IN A CONNECTED FITNESS PLATFORM, which is incorporated by reference in its entirety.

### BACKGROUND

The world of connected fitness is an ever-expanding one. This world can include a user taking part in an activity (e.g., running, cycling, lifting weights, and so on), other users also performing the activity, and other users doing other activities. The users may be utilizing a fitness machine (e.g., a treadmill, a stationary bike, a strength machine, a stationary rower, and so on), may be running, walking, bicycling, swimming, or performing another exercise activity or movement.

For example, the users may be performing activities that do not include an associated machine, such as running, strength training, yoga, stretching, hiking, climbing, and so on. These users can have wearable devices or mobile devices (e.g., heart rate monitors) that monitor the activity or performance of the users. The users can also perform exercise activities in front of or proximate to a user interface (e.g., a display or device) that presents content associated with the activities.

The user interface, whether a mobile device, a display device, or a display that is part of a machine, can provide or present interactive content to the users. For example, the user interface can present live or recorded classes, video tutorials of activities, leaderboards and other competitive or interactive features, progress indicators (e.g., via time, distance, and other metrics), and so on.

In some cases, the user may be part of a connected fitness platform, which provides and presents content for a single exercise activity (e.g., a platform that provides a single exercise machine, such as a rower) or many different exercise activities (e.g., a platform that is integrated with multiple exercise machines or exercise activities). In these cases, the platform may support and/or provide a large amount of content, including content that may be desired by some users, but not all users.

### BRIEF DESCRIPTION OF THE DRAWINGS

Embodiments of the present technology will be described and explained through the use of the accompanying drawings.

FIG. 1 is a block diagram illustrating a suitable network environment for users of an exercise system.

FIG. 2 is a block diagram illustrating hardware modules of a recommendation system.

FIG. 3 is a block diagram illustrating various modules of a connected fitness system that boosts content for users.

FIG. 4 is a diagram illustrating a class recommendation prediction.

FIG. 5 is a block diagram illustrating a class recommendation prediction that is personalized for a user.

FIG. 6 is a diagram that illustrates a visualization of an implementation of a boosting optimization algorithm.

FIG. 7 is a flow diagram illustrating an example method for recommending a class for a user of a connected fitness platform.

In the drawings, some components are not drawn to scale, and some components and/or operations can be separated into different blocks or combined into a single block for discussion of some of the implementations of the present technology. Moreover, while the technology is amenable to various modifications and alternative forms, specific implementations have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

### Overview

Various systems and methods that enhance an exercise activity performed by a user are described. In some embodiments, the systems and methods personalize content for members or users of a fitness platform, such as a connected fitness platform. The systems and methods, in some cases, utilize techniques to personalize an experience for a given user or member, such as via a user interface (e.g., a touchscreen on an exercise device or via their mobile device or wearable device), while balancing the usefulness of creating and providing large amounts of varied content via the platform for many different users, among other benefits.

For example, the systems and methods may surface relevant and timely recommendations of fitness or exercise classes to members of a connected fitness platform. Given a large and ever-expanding content library, such as classes/content that are created for certain themes appropriate during a certain timely event or theme, the systems and methods utilize various processes or methods to boost such content for users, such as content that is specific and/or personalized for single users or groups of users.

Thus, in various embodiments, the systems and methods personalize certain time-based content for users/members to enhance their experience when exercising or navigating a connected fitness platform. For example, the systems and methods may utilize machine learning models to solve an optimization problem, such as a non-linear optimization problem, when determining or predicting whether a boosted class will receive impressions (e.g., displays or presentations of the class) by users. In doing so, the systems and methods described herein may facilitate the boosting of content (e.g., timely content), in order to achieve/meet new or intended impression goals while minimizing any drops in conversion rates (due to users not being interested in the boosted content), among other benefits.

Various embodiments of the system and methods will now be described. The following description provides specific details for a thorough understanding and an enabling description of these embodiments. One skilled in the art will understand, however, that these embodiments may be practiced without many of these details. Additionally, some well-known structures or functions may not be shown or described in detail, so as to avoid unnecessarily obscuring the relevant description of the various embodiments. The terminology used in the description presented below is intended to be interpreted in its broadest reasonable manner, even though it is being used in conjunction with a detailed description of certain specific embodiments.

### Examples of a Suitable Exercise Platform

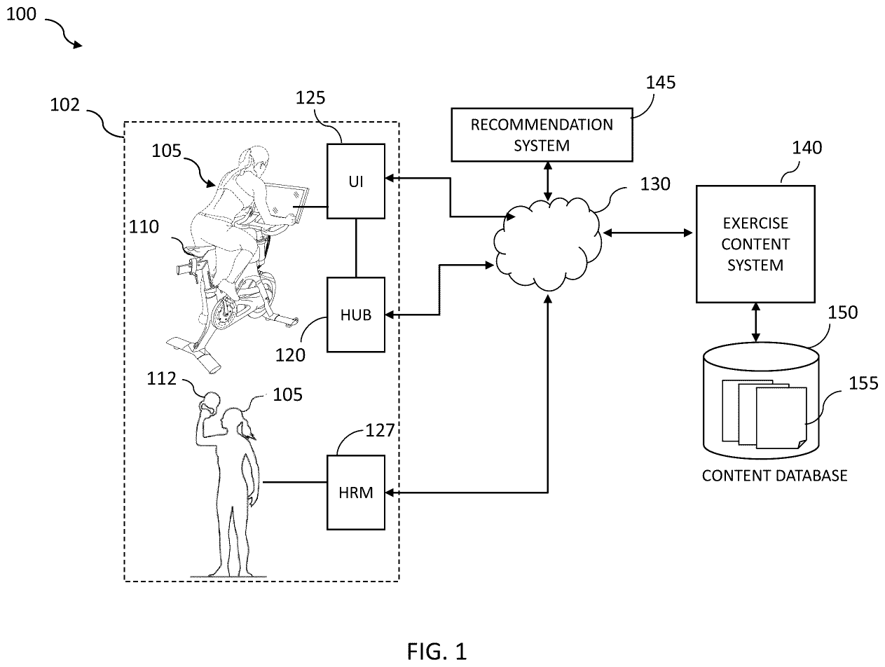

The technology described herein is directed, in some embodiments, to providing a user with an enhanced user experience (e.g., a personalize experience) when performing an exercise activity, such as an exercise activity as part of a connected fitness system or other exercise system. FIG. 1 is a block diagram illustrating a suitable network environment 100 for users of an exercise system.

The network environment 100 includes an activity environment 102, where a user 105 is performing an exercise activity, such as a cycling activity. In some cases, the user 105 can perform the activity with an exercise machine 110, such as exercise bicycle, a treadmill, a rowing machine, a stair climber, and so on. Further, the exercise activity performed by the user 105 can include a variety of different workouts, activities, actions, and/or movements not associated with a machine, such as movements associated with lifting weights 112 (as shown), stretching, doing yoga, pilates, rowing, running, cycling, jumping, sports movements (e.g., throwing a ball, pitching a ball, hitting, swinging a racket, swinging a golf club, kicking a ball, hitting a puck), and so on.

The exercise machine 110 can assist or facilitate the user 105 to perform the movements and/or can present interactive content to the user 105 when the user 105 performs the activity. For example, the exercise machine 110 can be a stationary bicycle, a stationary rower, a treadmill, a weight machine, or other machines. As another example, the exercise machine 110 can be a display device that presents content (e.g., streamed classes, dynamically changing video, audio, gaming activities, instructional content, and so on) to the user 105 during an activity or workout.

The exercise machine 110 includes a media hub 120 and a user interface 125. The media hub 120, in some cases, captures images and/or video of the user 105, such as images of the user 105 performing different movements, or poses, during an activity. The media hub 120 can include a camera or cameras, a camera sensor or sensors, or other optical sensors configured to capture the images or video of the user 105.

In some cases, the media hub 120 includes components configured to present or display information to the user 105. For example, the media hub 120 can be part of a set-top box or other similar device that outputs signals to a display, such as the user interface 125. Thus, the media hub 120 can operate to both capture images of the user 105 during an activity, while also presenting content (e.g., time-based or distance-based experiences, streamed classes, workout statistics, and so on) to the user 105 during the activity.

The user interface 125 provides the user 105 with an interactive experience during the activity. For example, the user interface 125 can present user-selectable options that identify live classes available to the user 105, pre-recorded classes available to the user 105, historical activity information for the user 105, progress information for the user 105, instructional or tutorial information for the user 105, and other content (e.g., video, audio, images, text, and so on), that is associated with the user 105 and/or activities performed (or to be performed) by the user 105.

In some cases, a heart rate monitor (HRM) 127 or other wearable device (e.g., smart watch, headphones, fitness trackers, and so on) can capture biometric information about the user 105, such as heart rate, movement information, sleep information, and so on. The HRM 127 can capture the user's heart rate and other information during machine-based activities and/or other activities, such as offline or class-based activities that do not utilize the exercise machine 110. In some cases, the exercise machine can include components configured to capture biometric information for the user 105, such as heart rate information.

The exercise machine 110, the media hub 120, and/or the user interface 125 can send or receive information over a network 130, such as a wireless network. Thus, in some cases, the user interface 125 is a display device (e.g., attached to the exercise machine 110), that receives content from (and sends information, such as user selections) an exercise content system 140 over the network 130. In other cases, the media hub 120 controls the communication of content to/from the exercise content system 140 over the network 130 and presents the content to the user via the user interface 125.

The exercise content system 140, located at one or more servers remote from the user 105, can include various content libraries (e.g., classes, movements, tutorials, and so on) and perform functions to stream or otherwise send content to the machine 110, the media hub 120, and/or the user interface 125 over the network 130.

A content database 150 stores content 155 (e.g., video files) that presents a pre-recorded class to a user. The content can include images, video, and other visual information that present the class, music and other audio information to be played during the activity, and various overlay or augmentation information that is presented along with the audio/video content. Further, the database 150 can include various content libraries (e.g., classes, movements, tutorials, and so on) associated with the content presented to the user during a selected experience.

As described herein, a recommendation system 145 can include various components or modules configured to boost recommendations and/or personalize a user or member experience within the environment 100. For example, the recommendation system 145 can determine or otherwise predict whether a user or group of users is likely to engage with certain content (e.g., a boosted or identified class or activity) and generate an output (e.g., re-rank a list of class personalized for the user or users) based on the prediction. Further details regarding the functionality of the recommendation system 145, including various prediction models and methods utilized to determine predictions for classes or other content, are described herein.

FIG. 1 and the components, systems, servers, and devices depicted herein provide a general computing environment and network within which the technology described herein can be implemented. Further, the systems, methods, and techniques introduced here can be implemented as special-purpose hardware (for example, circuitry), as programmable circuitry appropriately programmed with software and/or firmware, or as a combination of special-purpose and programmable circuitry. Hence, implementations can include a machine-readable medium having stored thereon instructions which can be used to program a computer (or other electronic devices) to perform a process. The machine-readable medium can include, but is not limited to, floppy diskettes, optical discs, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other types of media/machine-readable medium suitable for storing electronic instructions.

The network or cloud 130 can be any network, ranging from a wired or wireless local area network (LAN), to a wired or wireless wide area network (WAN), to the Internet or some other public or private network, to a cellular (e.g., 4G, LTE, or 5G network), and so on. While the connections between the various devices and the network 130 are shown as separate connections, these connections can be any kind of local, wide area, wire d, or wireless network, public or private.

Further, any or all components depicted in the Figures described herein can be supported and/or implemented via one or more computing systems, services (e.g., cloud instances), or servers. Although not required, aspects of the various components or systems are described in the general context of computer-executable instructions, such as routines executed by a general-purpose computer, e.g., mobile device, a server computer, or personal computer. The system can be practiced with other communications, data processing, or computer system configurations, including: Internet appliances, hand-held devices, wearable devices, or mobile devices (e.g., smart phones, tablets, laptops, smart watches), all manner of cellular or mobile phones, multi-processor systems, microprocessor-based or programmable consumer electronics, set-top boxes, network PCs, mini-computers, mainframe computers, AR/VR devices, gaming devices, and the like. Indeed, the terms “computer,” “host,” and “host computer,” and “mobile device” and “handset” are generally used interchangeably herein and refer to any of the above devices and systems, as well as any data processor.

Aspects of the system can be embodied in a special purpose computing device or data processor that is specifically programmed, configured, or constructed to perform one or more of the computer-executable instructions explained in detail herein. Aspects of the system may also be practiced in distributed computing environments where tasks or modules are performed by remote processing devices, which are linked through a communications network, such as a Local Area Network (LAN), Wide Area Network (WAN), or the Internet. In a distributed computing environment, program modules may be located in both local and remote memory storage devices.

Aspects of the system may be stored or distributed on computer-readable media (e.g., physical and/or tangible non-transitory computer-readable storage media), including magnetically or optically readable computer discs, hard-wired or preprogrammed chips (e.g., EEPROM semiconductor chips), nanotechnology memory, or other data storage media. Indeed, computer implemented instructions, data structures, screen displays, and other data under aspects of the system may be distributed over the Internet or over other networks (including wireless networks), or they may be provided on any analog or digital network (packet switched, circuit switched, or other scheme). Portions of the system may reside on a server computer, while corresponding portions may reside on a client computer such as an exercise machine, display device, or mobile or portable device, and thus, while certain hardware platforms are described herein, aspects of the system are equally applicable to nodes on a network. In some cases, the mobile device or portable device may represent the server portion, while the server may represent the client portion.

### Examples of the Recommendation System

As described herein, in some embodiments, the systems and methods perform various methods or operations to personalize a user experience within or associated with a connected fitness platform, such as by boosting content (e.g., an exercise class or series of classes associated with an event) for users based on predicted engagement metrics associated with the users selecting or otherwise engaging with the boosted content.

In some cases, the connected fitness platform maintains a dynamic inventory of ever-changing content, which can introduce various challenges when recommending classes to users via recommendations systems, such as system that attempt to surface timely or event-specific content that is also relevant to users of the platform. For example, the connected fitness platform can create and have a set of classes that is filmed and specifically relevant during a certain time. During this time, an owner or entity providing the platform may wish to reach a sizable audience with the timely content (e.g., to satisfy business or user viewership goals), while also preserving user engagement.

For example, timely content may be based on a new instructor being introduced to the platform, a new fitness discipline or type of class (e.g., a barre class or new combination of bootcamp activities) being introduced to the platform, a series of classes being filmed for a certain month of celebration (e.g., LatinX Heritage month), a new series of classes associated with a musician, artists, or celebrity is being introduced to the platform, and so on.

In some cases, naïvely surfacing or recommending this timely content can hurt user engagement goals, where the content is always recommended to all users. Thus, the platform may consider individual interests when selecting what recommendations of timely content users should receive. Thus, utilizing a recommendation system, which performs processes to determine recommendations based on a user's interests, can be utilized and/or enhanced to balance relevance and timeliness when generating recommendations of timely content to users of the connected fitness platform.

For example, the recommendation system 145 can selectively increase (or attempt to increase) impressions of certain classes, by “boosting” the impressions. An impression, as described herein, can relate to a display of content, such as within a display of recommended classes that is presented to a user via their home screen or other displayed interface.

The system 145 can facilitate the selection of timed boosts—boosts activated for a specified duration—of sets of classes, such as by re-ranking certain classes to move higher or lower within ranked lists of classes personalized for users, among other recommendation actions. Thus, the systems and methods described herein, via the recommendation system 145, can boost content in a personalized manner for the various users within the connected fitness environment 100, among other benefits.

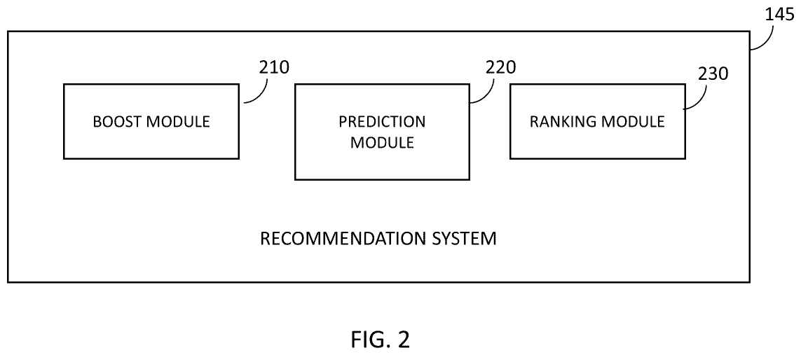

FIG. 2 is a block diagram illustrating hardware modules of the recommendation system 145. The modules and/or components of the recommendation system 145 can be implemented with a combination of software (e.g., executable instructions, or computer code) and hardware (e.g., at least a memory and processor). Accordingly, as used herein, in some example embodiments, a component/module is a processor-implemented component/module and represents a computing device having a processor that is at least temporarily configured and/or programmed by executable instructions stored in memory to perform one or more of the functions that are described herein. The recommendation system 145 includes a boost module 210, a prediction module 220, and a ranking module 230.

In some embodiments, the boost module 210 is configured and/or programmed to access one or more boost parameters for an exercise class available to multiple users of a connected fitness platform. For example, the boost module 210 can receive or otherwise access boost parameters for an exercise class, such as an identifier for the exercise class (or grouping of classes), a date or commencement to boost the class, a duration of the boost (e.g., an end parameter), a boost value (e.g., a fractional list or increase for the class), and so on.

In some embodiments, the prediction module 220 is configured and/or programmed to generate an engagement prediction score for the exercise class. For example, the prediction module 220 can apply and/or utilize one or more machine learning (ML) models to predict whether a user (or group of users) will engage with an exercise class (or other content) that is to be boosted within the platform. The engagement prediction score, as described herein, can represent a likelihood of engagement by the user using a variety of scales or metrics.

In some embodiments, the ranking module 230 is configured and/or programmed to update a list of class recommendations for each user of the multiple users of the connected fitness class based on the generated engagement prediction score and the one or more boost parameters. For example, the ranking module 230 may rank (or re-rank) a list of content that has been personalized for the user to include the boosted content based on the prediction (e.g., based on the engagement prediction score).

The ranking module 230, therefore, may determine a boosted prediction score (or boosted engagement prediction score) that combines or is based on the engagement prediction score and the boost parameters. The ranking module 230 may then compare the boosted prediction score for the connected fitness class to scores for other classes within the list recommended to the user and add or remove the connected fitness class to/from the list based on the comparison.

In some cases, a display module may be configured and/or programmed to present (or cause to present) the updated list of class recommendations to each user of the multiple users via display devices associated with each user. The display module may be part of the ranking module 230 and cause a UI associated with the users (e.g., the UI 125 or a UI associated with the exercise machine 110, a smart watch, mobile device, and so on) to display graphical elements that represent the recommended classes.

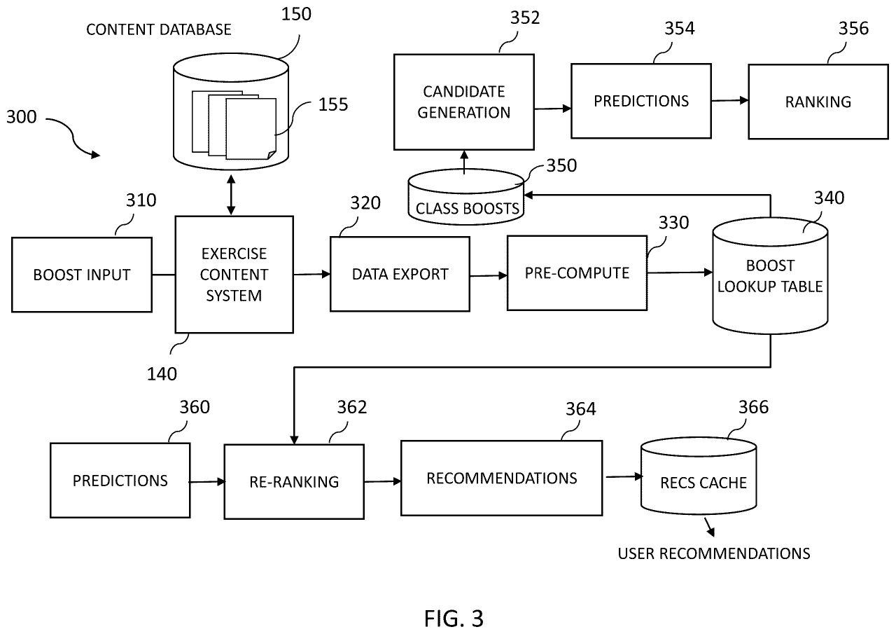

FIG. 3 illustrates an example data flow 300 between the various components/modules of the recommendation system 145 within the connected fitness platform. For example, the data flow 300 depicts how boosts are applied in both batch form (e.g., in an offline mode where a boost is applied to multiple classes) and in a real-time or dynamic manner (e.g., in an online mode).

Boost input 310 is received via a web portal, such as a webpage that is part of a content management system (CMS), such as a CMS associated with the exercise content system 140, and accessible via one or more administrators of a platform (e.g., an external team such as marketing team that wishes to boost a certain set of classes). As described herein, the boost input 310 can include parameters that identify a set of classes, a date on which the boost starts, a boost value, a date on which the boost ends, and other parameters. The boost value, for example, can indicate a fractional lift in impressions, compared to a baseline number or percentage of impressions (e.g., a boost value of +1.0 indicates a 100% desired lift in impressions for an identified class within a time window).

The parameters within the boost input 310 are exported via a data export module 320 or similar function, which sends the parameters to a pre-computation module 330. The pre-computation module 330 may periodically (e.g., hourly or daily) collate boosts for a given time window (e.g., a day), and determines a boost per class within the content database 150 or other class library. A boost lookup table (LUT) receives and stored the boost for each class and facilitates the generation of recommendations in various modes of operation.

For example, in the online mode, a class boost database 350, implemented as a Redis module or cache (or other dynamic library) is accessed by a candidate generation module 352 (e.g., part of the recommendation system 145), which can generate predictions for classes (e.g., via an applied ML prediction model), as described herein, such as engagement prediction scores for each of the classes. The recommendation system 145 may then generate or update a ranking of classes for a user or user based on the generated predictions.

As another example, in the offline or batch mode, the system 145 accesses generated offline model predictions 360 and performs a re-ranking process 362 based on the received boosts (e.g., via the boost LUT 340). The re-ranking process 362 may apply various boosting or prediction algorithms or frameworks, as described herein, and generate recommendations 364, which may be output to and stored in a recommendations cache 366. The platform may then access the recommendations cache 366 to present, display, surface, or otherwise provide the recommendations to users of the platform.

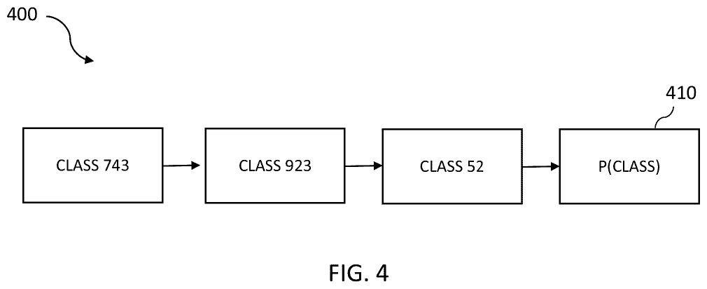

The recommendation system 145 can utilize various approaches when generated recommendations (e.g., via the batch mode) for users of boosted classes. As a first example, the system 145 may employ multiplicative boosts, where the system 145, (e.g., via the prediction module 220 and/or the ranking module 230) multiplies a model-generated prediction score (e.g., an engagement prediction score) with the boost value, and sorts the results based on the new scores. Such an approach, as depicted in the class sequence 400 of FIG. 4, facilitates a prediction or recommendation of a new class 410 (e.g., a timely class) that may already be highly ranked for a user or users.

Table 1 depicts results of an A/B test utilizing the multiplicative approach, where impressions were increased but various issues may arise in certain cases.

TABLE 1
Boosting Experiment Results using a Multiplicative Approach

| | | Target | Actual | Change in |
|---|---|---|---|---|
| | | Impressions | Impressions | Conversion |
| Boost | Timing | Lift | Lift | Rate |
| Expt. 1 | Nov. 8-15, 2021 | 3x | 30x | −9.0% |
| Expt. 2 | Feb. 8-11, 2022 | 3x | 111x | 0% |
| Expt. 3 | Feb. 15-18, 2022 | 1.5x | 1.9x | −11.1% |

For example, in some cases, the system 145 may not be able to control an actual or realized lift in impressions (e.g., see experiment 1, where a multiplier algorithm led to a 30× actual lift in impressions when the desired lift was 3×). Further, the system 145 may realize a drop in engagement with our recommendations (e.g., a negative conversion rate) during the boosting period. Again, in experiment 1, there is a conversion drop of 9%. Thus, some issues, such as the approach selecting too many users display the boosted classes and/or the approach selecting users having lower interest in these classes, may limit the use of the multiplicative approach in certain cases.

The recommendation system 145, therefore, may utilize an algorithmic boost approach to mitigate certain issues in the simpler, multiplicative, approach. As described herein, the recommendation system 145 may utilize ML models, such as a long short-term memory neural network (e.g., LSTMNet) or another recurrent neural network (RNN), a deep learning recommendation model (DLRM), and/or other ML models that utilize implicit or explicit signals when determining recommendations for users based on user preferences and/or user activities.

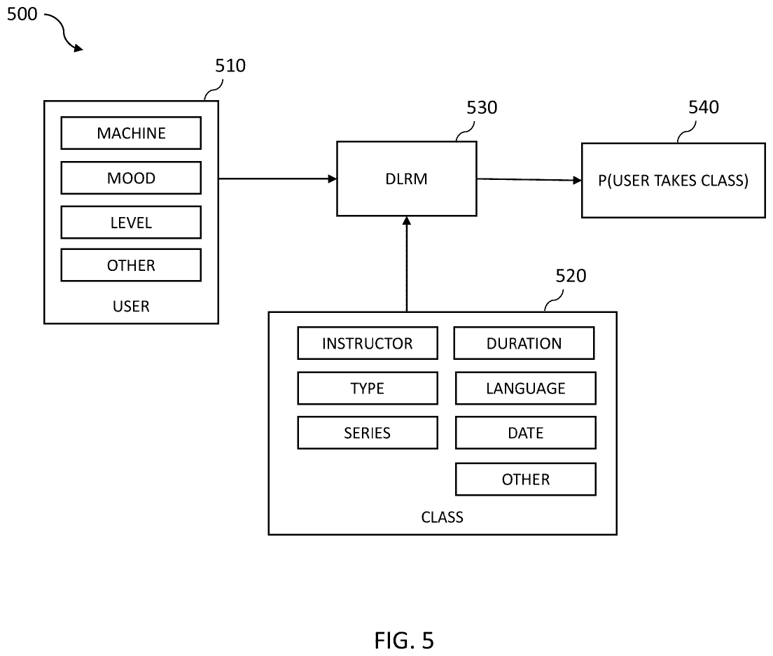

For example, FIG. 5 is a block diagram 500 illustrating a class recommendation prediction that is personalized for a user. The recommendation system 145, as depicted, can implement a deep learning recommendation model 530, which receives user input 510 (e.g., user preference information) and class input 520 (e.g., class parameters or characteristics, and generates a prediction 540 that the user will take or otherwise engage with the class.

In some cases, the user input 510 can include machine information (e.g., what exercise machine or class type is desired by the user), mood information (e.g., the user is excited or in a state of recovery), level information (e.g., the user is a beginner to the platform or to a specific exercise or class), and/or other information associated with the user (e.g., the time of day, the number of workouts performed in a certain time period).

In some cases, the class input 520 can include instruction information (e.g., the name of the instructor), the class type (e.g., the exercise activities performed during the class), series information (e.g., the class is part of a set or stack of classes), the duration of the class (e.g., the class is 20 minutes long), the language of instruction, the date the class was released (e.g., the timeliness of the class), and/or other information associated with the class (e.g., the music within the class, the level of the class, and so on).

In some embodiments, such as in the batch mode, the system 145 generates recommendations for some or all users at the same time. Thus, the system 145 can selectively distribute impressions of a desired set of classes across users in a more optimal or enhanced manner. For example, the system 145 can apply a constrained optimization technique to minimize a conversion drop while realizing a target lift in impressions.

The system 145 can act to increase impressions at a granularity of a collection of content (e.g., and not individually per class), where the content to be boosted is a boosting set or boosted set of content. The system 145 may consider a user viewing a class (e.g., via a recommended classes interface) as a general impression, and, therefore, may consider a user viewing a class from a boosting set as an impression for the boosting set.

Thus, the system 145 may consider an occurrence of boosting when an aggregate number of impressions across the boosting set has met a target number of impressions. For example, classes are represented via cₘ, m∈{1, 2, . . . . M}, users are represented via un, n∈{1, 2, . . . . N}, and a collection of boosting sets is represented via Bₗ, l∈{1, 2, . . . . L}, where the number of impressions may be boost times over what a specified set of classes would normally obtain as impressions without boosting.

For each user and class pair, the system 145 determines an associated prediction score p_{m,n}, which indicates a likelihood of the user n to convert (e.g., view or otherwise cause an impression) on the class m (e.g., the engagement prediction score). As described herein, the system 145 may utilize one or more ML models to generate the prediction score for a user and class pair. In some cases, the system 145 may generate recommendations for the user using a weighted random sampling (without replacement) of the generated predictions, using the prediction scores as the weights. Such sampling may ensure freshness of the recommendations, a variety of options for the user, and so on.

The approach may be performed as follows. For each boost set Bₗ, the approach may calculate an estimated number of user impressions (Iₗ) without boosting. This is calculated as the number of classes in Bₗ which appear in the top-K recommendations for the users, where impression-per-user is proportional to the model score of the class divided by the sum of scores in the top-K, due to the weighted random selection performed. See equation 1, as follows:

$${I}_{l} = {\sum }_{n = 1}^{N} ( \frac{{\sum }_{k = 1}^{K} 1 ( {c}_{k} , {B}_{l} ) {p}_{n , k}}{{\sum }_{k = 1}^{K} {p}_{n , k}} ) \qquad ( Equation 1 )$$

where 1 (cₖ, Bₗ)=1, if cₖ∈Bₗ, otherwise 0

Carefully selecting recommendations in the top-K for each user, the system 145 lifts the number of estimated impressions for the boost set to a value of boostₗ·Iₗ, where there is an indicator variable a_{n,m} such that a_{n,m}=1 when the class is chosen to be in the user's top-K recommendations, and a_{n,m}=0 for all other cases. Further, there is a boost variable b_{n,m}, which contains an exact boost factor for each class. Then the approach determines an optimization problem (which solves for a_{n,m} and b_{n,m}) as Equation 2, shown below:

$$Maximize {\sum }_{n = 1}^{N} {\sum }_{m = 1}^{M} {a}_{n , m} {p}_{n , m} ( e . g . , maximize conversion probability ) \qquad ( Equation 2 )$$

$$s . t . : {\sum }_{n = 1}^{N} ( \frac{{\sum }_{k ∈ {B}_{l}} {a}_{n , k} {b}_{n , k} {p}_{n , k}}{{\sum }_{m = 1}^{M} {a}_{n , m} {p}_{n , m}} ) \geq {boost}_{l} \cdot {I}_{l} , for each {B}_{l}$$

$${\sum }_{m = 1}^{M} {a}_{n , m} = K , for each n ( e . g . , ensure that only K items are selected for each user )$$

$${b}_{n , m} = 1. , for each {c}_{m} ∉ {B}_{l} ( e . g . , ensure that boost values only apply to boosting sets , and not to other classes )$$

$${a}_{n , m} ∈ \{ 0 , 1 \} ( e . g . , the selection variable is binary )$$

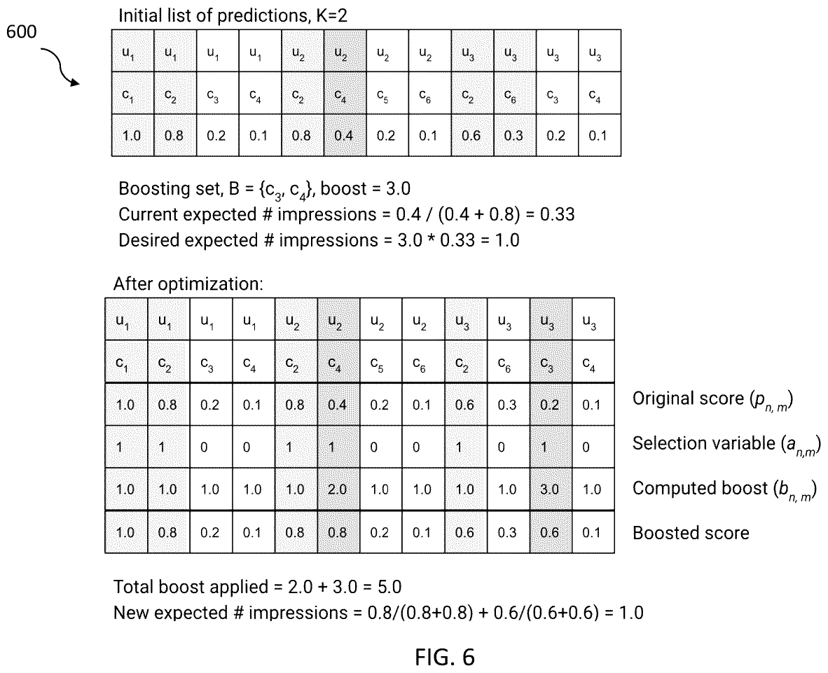

FIG. 6 depicts a visualization 600 of an implementation of the boosting optimization algorithm (Equation 2) using three users and a small set of classes. Such an implementation realizes a non-linear optimization problem (which is not provably convex), where various assumptions (e.g., that all users view their recommendations may underly the formulation. However, the approach may apply approximations to modify the problem to one of a convex nature.

For example, since a primary source of non-linearity is the selection variable, one approximation can be to pre-process the scores to realize K high-scored items (with the remaining items being low-scoring), thereby removing the selection variable. The approach may use a “soft-top-K” function, which is based on the index (k) of the score in the sorted list of scores, as follows:

$$soft\_top \_k ( k ) = exp ( w \cdot min ( 0 , K - k ) ) \qquad ( Equation 3 )$$

The shape of a function for K=50 and different selections of w are shown below. When choosing w, the approach may prevent scores from decaying too quickly, because classes to be boosted may be outside the top-K and will require a finite score to be boosted. Also, slow decay may also lead to many non-boosted classes outside the top-K having a finite chance of being shown in user recommendations. Thus, during implementation, the approach found a value of w=0.25 provides a good balance of decay speed.

Next, the approach selects, via weighted sampling from an entire set (no longer just the top-K), the K highest scored items are much more likely to be picked up than the remaining low-scoring ones, because of how much their scores have decayed. At the same time, the classes from the boosted set may be in the tail and have a finite score (>0.0), which gives them a chance of being boosted. Using this simplification or approximation, the approach can remove the selection variable a_{n,m}. The problem formulation becomes the following, as shown in Equation 4:

$$Minimize {\sum }_{n = 1}^{N} {\sum }_{k ∈ {B}_{l}} {b}_{n , k} ( e . g . , minimizing the amount of boosting has the effect of maximizing the sum of recommendation scores ) \qquad ( Equation 4 )$$

$$s . t . : , {\sum }_{n = 1}^{N} ( \frac{{\sum }_{k ∈ {B}_{l}} {b}_{n , k} {p}_{n , k}}{{\sum }_{m = 1}^{M} {p}_{n , m}} ) \geq {boost}_{l} \cdot {I}_{l} , for each {B}_{l} ( e . g . , an estimated number of impressions exceeds the target ) .$$

The approach utilizes an additional approximation, and assumes that applying the boost multiplier to a small set of classes does not significantly alter the total sum of scores, or

$${\sum }_{m = 1}^{M} {p}_{n , m} i . e . {\sum }_{m = 1}^{M} {b}_{n , m} {p}_{n , m} ≃ {\sum }_{m = 1}^{M} {p}_{n , m} ,$$

where b_{n,m}=1.0 for most classes. Such an approximation enables the denominator to be held constant in the constraint.

Thus, the approach can reformulate to a single objective function that can be optimized by using a highly parallelizable gradient descent (or SGD), which can be solved at scale using a unified analytics engine (e.g., Apache Spark), even for millions of users and thousands of classes per user. This single objective function is shown as Equation 5, as follows:

$$Cost = {\sum }_{l ∈ L} {( {\sum }_{n = 1}^{N} ( \frac{{\sum }_{k ∈ {B}_{l}} {b}_{n , k} {p}_{n , k}}{{\sum }_{m = 1}^{M} {p}_{n , m}} ) - {boost}_{l} \cdot {I}_{l} )}^{2} + β {\sum }_{n = 1}^{N} {\sum }_{k ∈ {B}_{l}} {b}_{n , k}^{2} \qquad ( Equation 5 )$$

Minimizing the first term, the approach gets close to the target number of impressions. The second term helps control for very high boost values—it is simply a regularization term. This function is differentiable, and the first order derivative δCost/δb_{n,k} can be derived analytically. This optimization can then be solved completely in parallel via an analytics engine, which can compute the derivative for each boosted class-user combination and apply an update function b_{n,k}=b_{n,k}−ηδCost/δb_{n,k}, where n is a tunable learning rate. Table 2 depicts a comparison of the described approaches:

TABLE 2

| | | | Target | Actual | Change in |
|---|---|---|---|---|---|
| | | | Impressions | Impressions | Conversion |
| Boost | Timing | Variation | Lift | Lift | Rate |
| Expt. 4 | May 24-31, 2022 | MB | 2x | 22.1x | −17.9% |
| | | AB | | 1.4x | 0% |
| Expt. 5 | June 22-30, 2022 | MB | 2x | 1.1x | −5.7% |
| | | AB | | 1.1x | −2.2% |

As shown in Table 2, the algorithmic boost approach (e.g., AB) shows a minimized drop in the conversion rate with respect to a controlled actual impressions lift (versus a target impressions lift)

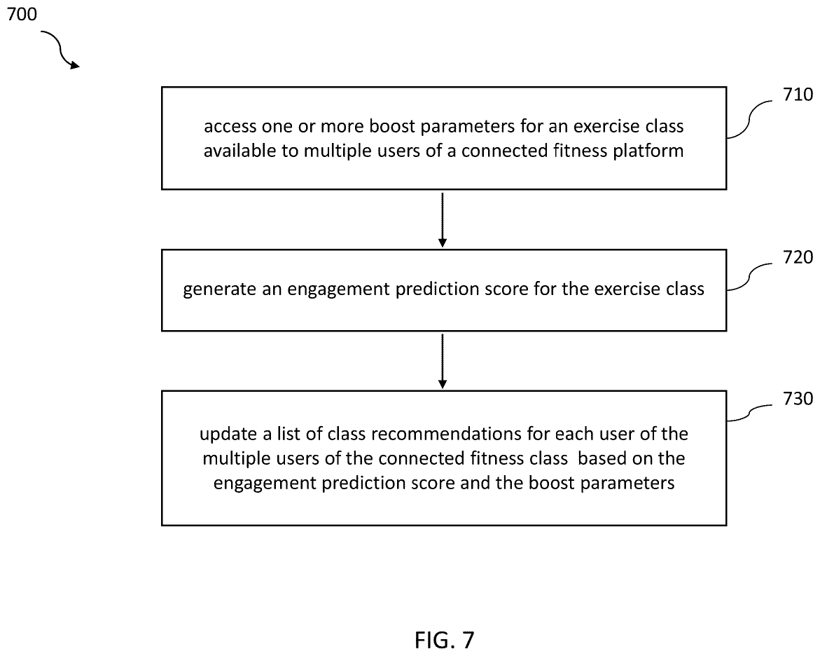

Thus, as described herein, the recommendation system 145 may utilize deep learning prediction models to determine predictions for boosting classes for users within their personalized recommendations. FIG. 7 is a flow diagram illustrating an example method 700 for recommending a class for a user of a connected fitness platform. The method 700 may be performed by the recommendation system 145 and, accordingly, is described herein merely by way of reference thereto. It will be appreciated that the method 700 may be performed on any suitable hardware.

In operation 710, the recommendation system 145 accesses one or more boost parameters for an exercise class available to multiple users of a connected fitness platform. For example, the boost module 210 can receive or otherwise access boost parameters for an exercise class, such as an identifier for the exercise class (or grouping of classes), a date or commencement to boost the class, a duration of the boost (e.g., an end parameter), a boost value (e.g., a fractional list or increase for the class), and so on.

In operation 720, the recommendation system 145 generates an engagement prediction score for the exercise class. For example, the prediction module 220 can apply and/or utilize one or more machine learning (ML) models to predict whether a user (or group of users) will engage with an exercise class (or other content) that is to be boosted within the platform. The engagement prediction score, as described herein, can represent a likelihood of engagement by the user using a variety of scales or metrics.

In operation 730, the recommendation system 145 updates a list of class recommendations for each user of the multiple users of the connected fitness class based on the generated engagement prediction score and the one or more boost parameters. For example, the ranking module 230 may rank (or re-rank) a list of content that has been personalized for the user to include the boosted content based on the prediction (e.g., based on the engagement prediction score). The ranking module 230 may generate a list of recommendations (e.g., a list of classes) based on a combination of the prediction scores and the boost values, as described herein.

Thus, in various embodiments, the recommendation system 145 realizes and solves an optimization problem, such as a non-linear optimization problem, when determining or prediction whether a boosted class will receive impressions by users. For example, the system 145 may access a boost value applied to an exercise class available to multiple users of the connected fitness platform within a time window, determine a solution to a non-linear optimization problem that includes the multiple users and the exercise class as variables to generate an engagement prediction for each user of the multiple users; and determine a recommendation of the exercise class based on the generated engagement prediction for each user of the multiple users.

In doing so, the systems and methods described herein may facilitate the boosting of content (e.g., timely content) to users that realizes new or intended impression goals while minimizing any drops in conversion rates (due to users not being interested in the boosted content), among other benefits.

### Example Embodiments of the Technology

The technology described herein can include some or all of the following embodiments.

In some embodiments, a system includes multiple hardware modules executable by a processor of a computing system within a connected fitness platform, including a boost module that accesses one or more boost parameters for an exercise class available to multiple users of the connected fitness platform, a prediction module that generates an engagement prediction score for the exercise class, and a ranking module that updates a list of class recommendations for each user of the multiple users of the connected fitness class based on the generated engagement prediction score and the one or more boost parameters.

In some cases, the system may include a display module that presents the updated list of class recommendations to each user of the multiple users via display devices associated with each user.

In some cases, the engagement prediction score is an indication of a likelihood that a user of the multiple users views the exercise class.

In some cases, the prediction module generates a unique engagement prediction score for each user of the multiple users with the exercise class.

In some cases, the prediction module applies a constrained optimization technique to determine a boosted prediction score for the exercise class.

In some cases, the prediction module solves a non-linear optimization problem to determine a boosted prediction score for the exercise class.

In some cases, the prediction module determines the engagement prediction score using a deep learning recommendation model.

In some cases, the one or more boost parameters include a boost value for the exercise class.

In some cases, the one or more boost parameters include a boost value for the exercise class and a time window within the boost value is to be applied to the exercise class.

In some cases, the multiple users are associated with exercise bicycles, and the exercise class is a live cycling class.

In some cases, the multiple users are associated with treadmills, and the exercise class is a live running class.

In some cases, the multiple users are associated with wearable devices, and the exercise class is a live running class.

In some embodiments, a method performed by a recommendation system of a connected fitness platform includes accessing one or more boost parameters for an exercise class available to multiple users of the connected fitness platform, generating an engagement prediction score for the exercise class, and updating a list of class recommendations for each user of the multiple users of the connected fitness class based on the generated engagement prediction score and the one or more boost parameters.

In some embodiments, a method includes accessing a boost value applied to an exercise class available to multiple users of the connected fitness platform within a time window, determining a solution to a non-linear optimization problem that includes the multiple users and the exercise class as variables to generate an engagement prediction for each user of the multiple users, and determining a recommendation of the exercise class based on the generated engagement prediction for each user of the multiple users.

### CONCLUSION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or”, in reference to a list of two or more items, covers all of the following interpretations of the word: any of the items in the list, all of the items in the list, and any combination of the items in the list.

The above detailed description of embodiments of the disclosure is not intended to be exhaustive or to limit the teachings to the precise form disclosed above. While specific embodiments of, and examples for, the disclosure are described above for illustrative purposes, various equivalent modifications are possible within the scope of the disclosure, as those skilled in the relevant art will recognize.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further embodiments of the disclosure.

These and other changes can be made to the disclosure in light of the above Detailed Description. While the above description describes certain embodiments of the disclosure, and describes the best mode contemplated, no matter how detailed the above appears in text, the teachings can be practiced in many ways. Details of the technology may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

From the foregoing, it will be appreciated that specific embodiments have been described herein for purposes of illustration, but that various modifications may be made without deviating from the spirit and scope of the embodiments. Accordingly, the embodiments are not limited except as by the appended claims.

What is claimed is:

1. A system, comprising:
multiple hardware modules executable by a processor of a computing system within a connected fitness platform, the hardware modules including:
a boost module that accesses one or more boost parameters for an exercise class available to multiple users of the connected fitness platform;
a prediction module that generates an engagement prediction score for the exercise class; and
a ranking module that updates a list of class recommendations for each user of the multiple users of the connected fitness class based on the generated engagement prediction score and the one or more boost parameters.

2. The system of claim 1, further comprising:
a display module that presents the updated list of class recommendations to each user of the multiple users via display devices associated with each user.

3. The system of claim 1, wherein the engagement prediction score is an indication of a likelihood that a user of the multiple users views the exercise class.

4. The system of claim 1, wherein the prediction module generates a unique engagement prediction score for each user of the multiple users with the exercise class.

5. The system of claim 1, wherein the prediction module applies a constrained optimization technique to determine a boosted prediction score for the exercise class.

6. The system of claim 1, wherein the prediction module solves a non-linear optimization problem to determine a boosted prediction score for the exercise class.

7. The system of claim 1, wherein the prediction module determines the engagement prediction score using a deep learning recommendation model.

8. The system of claim 1, wherein the one or more boost parameters include a boost value for the exercise class.

9. The system of claim 1, wherein the one or more boost parameters include a boost value for the exercise class and a time window within the boost value is to be applied to the exercise class.

10. The system of claim 1, wherein the multiple users are associated with exercise bicycles, and the exercise class is a live cycling class.

11. The system of claim 1, wherein the multiple users are associated with treadmills, and the exercise class is a live running class.

12. The system of claim 1, wherein the multiple users are associated with wearable devices, and the exercise class is a live running class.

13. A method performed by a recommendation system of a connected fitness platform, the method comprising:
accessing one or more boost parameters for an exercise class available to multiple users of the connected fitness platform;
generating an engagement prediction score for the exercise class; and
updating a list of class recommendations for each user of the multiple users of the connected fitness class based on the generated engagement prediction score and the one or more boost parameters.

14. The method of claim 13, further comprising:
presenting the updated list of class recommendations to each user of the multiple users via display devices associated with each user.

15. The method of claim 13, wherein the engagement prediction score is an indication of a likelihood that a user of the multiple users views the exercise class.

16. The method of claim 13, wherein the prediction module generates a unique engagement prediction score for each user of the multiple users with the exercise class.

17. The method of claim 13, wherein the prediction module applies a constrained optimization technique to determine a boosted prediction score for the exercise class.

18. The method of claim 13, wherein the prediction module solves a non-linear optimization problem to determine a boosted prediction score for the exercise class.

19. The method of claim 13, wherein the prediction module determines the engagement prediction score using a deep learning recommendation model.

20. A non-transitory computer-readable medium whose contents, when executed by a recommendation system of a connected fitness platform, causes the recommendation system to perform a method, the method comprising:
accessing a boost value applied to an exercise class available to multiple users of the connected fitness platform within a time window;
determining a solution to a non-linear optimization problem that includes the multiple users and the exercise class as variables to generate an engagement prediction for each user of the multiple users; and
determining a recommendation of the exercise class based on the generated engagement prediction for each user of the multiple users.
